(19) United States
(12) Patent Application Publication
(10) Pub. No.: US 2008/0167720 A1
(43) Pub. Date: Jul. 10, 2008

(54) Expandable vertebral body replacement device

(75) Inventor: Anthony J. Melkent, Memphis, TN (US)

Correspondence Address: KRIEG DEVAULT LLP, ONE INDIANA SQUARE, SUITE 2800, INDIANAPOLIS, IN, 46204-2709, US

(73) Assignee: Warsaw Orthopedic, Inc.

(21) Appl. No.: 11/650,746

(22) Filed: Jan. 8, 2007

(51) Int. Cl. A61F 2/44; A61B 17/70

(52) U.S. Cl. 623/17.16; 606/279

(57) ABSTRACT

An expandable vertebral replacement device and method of using the same is disclosed that allows surgeons to support two adjacent vertebrae after an intermediate vertebra or a portion of an intermediate vertebra has been removed for the spine. The expandable vertebral replacement device includes a first replacement body, a second replacement body and a collar. The second replacement body includes a projecting portion that is telescopically received within an axial passage defined by the first replacement body. The collar is used to force the collet of the first replacement body into clamping engagement with the projecting portion of the second replacement body to lock the expandable vertebral replacement device at a select height.

### CROSS REFERENCE TO RELATED APPLICATIONS

The subject application is related to a U.S. patent application entitled “Ratcheting Expandable Corpectomy/Vertebrectomy Cage” filed on the same day as the subject patent application and referred to under matter number P25985.00 and attorney docket number 31132.582, the contents of which are hereby incorporated by reference in their entirety. Each of these applications is assigned to a common assignee.

### TECHNICAL FIELD

The present invention relates generally to the field of surgical devices and methods, and more particularly relates to surgical devices and methods for use in the replacement of one or more vertebral bodies or other orthopedic structures.

### BACKGROUND

It is sometimes necessary to remove one or more vertebrae, or a portion of the vertebrae, from the human spine in response to various pathologies. For example, one or more of the vertebrae may become damaged as a result of tumor growth, or may become damaged by trauma or other events. Excision of at least a portion of the vertebral body is typically referred to as a corpectomy procedure. An implant may be placed within the intervertebral space between the remaining vertebrae to provide structural support and stability to the spine. In some instances, the implant inserted between the vertebrae is designed to facilitate fusion between the remaining vertebrae. In other instances, the implant is designed to replace the function of the excised vertebral body and discs.

Many implants are known in the art for use in a corpectomy procedure. One class of implants is configured to directly replace the removed vertebra or vertebrae. Another class of implants is configured for insertion into the intervertebral space in a collapsed state and then expanded once properly positioned within the intervertebral space between the remaining adjacent vertebrae. The use of expandable implants may be advantageous since a smaller incision is required to insert the implant into the intervertebral space. Additionally, expandable implants may assist with restoring proper loading to the spinal anatomy and achieving more secure fixation of the implant. Implants which include insertion and expansion members that have a narrow profile may also provide clinical advantages.

In some circumstances, it is desirable to have vertebral endplate contacting surfaces that effectively spread loading across the vertebral endplates. Implants may also include a member for maintaining the desired positions, and in some situations, being capable of collapsing. Fusion implants including one or more openings may also be advantageous because they allow for vascularization and bone growth through all or a portion of the implant. Expandable implants may also be useful in replacing long bones or portions of appendages such as the legs and arms, or a rib or other bone structures that are generally longer than wide. Examples include, but are not limited to, a femur, tibia, fibula, humerus, radius, ulna, phalanges, clavicle, and any of the ribs.

There remains a need for improved surgical devices and methods for use in the replacement of one or more vertebral bodies or other orthopedic structures.

### SUMMARY

The present relates generally to the field of surgical devices and methods, and more particularly relates to surgical devices and methods for use in the replacement of one or more vertebral bodies or other orthopedic structures. While the actual nature of the invention covered herein can only be determined with reference to the claims appended hereto, certain forms of the invention that are characteristic of the preferred embodiments disclosed herein are described briefly as follows.

In one form of the present invention, an expandable vertebral replacement device is provided, including a first replacement body having a collet, a second replacement body including a projecting portion telescopically received within the collet, and a collar positioned about the first replacement body for engaging the collet to the second replacement body to thereby secure the second replacement body in a fixed position relative to the first replacement body.

In another form of the present invention, an expandable vertebral replacement device is provided, including a first cylindrical replacement body having an upper portion and a lower portion defining at least a partially hollow interior area wherein the upper portion includes a collet, a second cylindrical replacement body including a projecting portion positioned within the collet of the first cylindrical replacement body, and a collar positioned about the upper portion of the first cylindrical replacement body for fixedly engaging the collet to the projecting portion of the second cylindrical replacement body to maintain the second cylindrical replacement body in a fixed position relative to the first cylindrical replacement body.

In another form of the present invention, a method is provided for supporting two adjacent vertebrae, including placing an expandable vertebral replacement device between the adjacent vertebrae, positioning an external distractor into engagement with opposite end portions of the expandable vertebral replacement device, expanding the expandable vertebral replacement device along a longitudinal axis to a select height between the first and second vertebrae with the external distractor, and rotating a collar portion of the expandable vertebral replacement device about the longitudinal axis to lock the expandable vertebral replacement device at the select height.

It is one object of the present invention to provide improved surgical devices and methods for use in the replacement of one or more vertebral bodies or other orthopedic structures. Further objects, features, advantages, benefits, and aspects of the present invention will become apparent from the drawings and description contained herein.

### BRIEF DESCRIPTION OF THE DRAWINGS

The components in the figures are not necessarily to scale, emphasis instead being placed upon illustrating the principles of the invention. Moreover, in the figures, like reference numerals designate corresponding parts throughout the different views.

FIG. 1 is a perspective view of an expandable vertebral replacement device according to one form of the present invention.

FIG. 2 is a perspective view of a first replacement body of the expandable vertebral replacement device illustrated in FIG. 1.

FIG. 3 is a perspective view a second replacement body of the expandable vertebral replacement device illustrated in FIG. 1.

FIG. 4 is a cross-sectional view of the expandable vertebral replacement device illustrated in FIG. 1.

FIG. 5 is a flow chart illustrating a method according to one form of the present invention for inserting an expandable vertebral replacement device within an intervertebral space to separate adjacent vertebrae.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

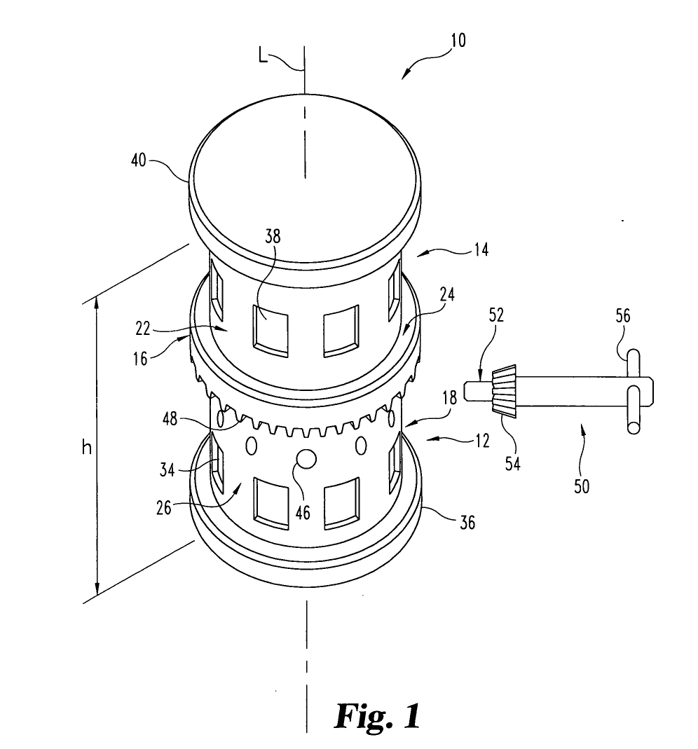

Referring to FIG. 1, shown therein is a representative expandable vertebral replacement device 10 according to one form of the present invention. The vertebral replacement device 10 may be used to maintain a fixed space between two vertebrae of the spine following removal of at least a portion of an intermediate vertebra and the adjacent disc material. In one embodiment of the invention, the vertebral replacement device 10 is used in association with the lumbar region of the spine. However, it is also contemplated that the vertebral replacement device 10 may be used in association with other regions of the spine, including the cervical and thoracic regions. It is further contemplated that the vertebral replacement device 10 may be used in association with other bone structures outside of the spine.

In the illustrated embodiment of the invention, the expandable vertebral replacement device 10 extends generally along a longitudinal axis L and includes a first replacement body 12, a second replacement body 14 and a collar 16. In one embodiment, the first and second replacement bodies 12, 14 each have a generally cylindrical configuration. However, as will be discussed below, other shapes and configurations are also contemplated. The second replacement body 14 is sized and configured to be telescopically received within an axial passage in the first replacement body 12. Prior to locking the replacement bodies into position, the position of the second replacement body 14 is capable of being telescopically adjusted relative to the first replacement body 12 generally along the longitudinal axis L, thereby allowing the overall height h or axial length of the expandable vertebral replacement device 10 to be adjusted to a select height or axial length. The collar 16 is utilized to tighten or clamp the second replacement body 14 in a fixed or locked state relative to the first replacement body 12.

In the illustrated embodiment of the invention, each of the replacement bodies 12 and 14 has a tubular configuration defining an axial passage or hollow interior. However, in another embodiment, only the first replacement body 12 is provided with a tubular configuration defining an axial passage, with the second replacement body being provided with a substantially solid configuration. Moreover, while the first replacement body 12 has been illustrated as having a tubular configuration defining an axial passage for telescopically receiving the second replacement body 14, in another embodiment of the invention, the second replacement body 14 may be provided with a tubular configuration defining an axial passage for telescopically receiving the first replacement body 12.

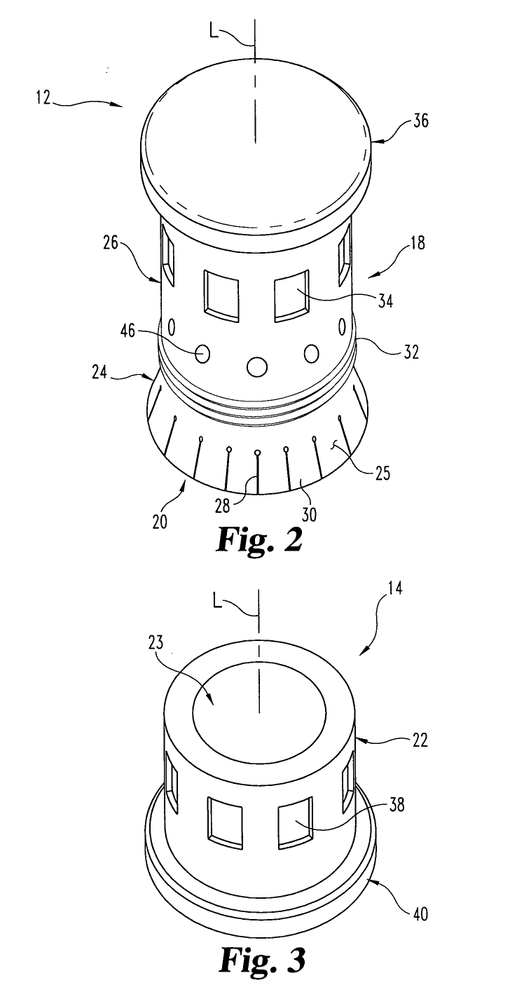

Referring to FIGS. 2 and 3, shown therein is the first replacement body 12 and the second replacement body 14 according to respective embodiments of the present invention. The first replacement body 12 includes a collet 18 having a generally cylindrical or tubular configuration defining an axial passage 20 that is sized somewhat larger than the outer cross section of a projecting portion or stem 22 defined by the second replacement body 14. In the illustrated embodiment, the projecting portion 22 of the second replacement body 14 also has a generally cylindrical or tubular configuration. The projecting portion 22 of the second replacement body 14 is sized and configured to be telescopically displaced within the axial passage 20 in the first replacement body 12 when the collar 16 is not securing the second replacement body 14 in position relative to the first replacement body 12. Accordingly, the overall height h of the expandable vertebral replacement device 10 can be precisely adjusted to fit between respective vertebrae.

In the illustrated embodiment, the inner surface of the collet 18 extending about the axial passage and the outer surface of the projecting portion 22 are substantially smooth and devoid of any surface projections (e.g., threads) to facilitate relatively uninhibited axial sliding of the projecting portion 22 within the axial passage 20 when the collar 16 is not securing the second replacement body 14 in position relative to the first replacement body 12. In this manner, the expandable vertebral replacement device 10 is configured to allow for infinite adjustment to the overall height h of the device. However, in other embodiments of the invention, the inner surface defined by the conical portion 24 of the collet 18 and/or the outer surface defined by the projecting portion 22 may be provided with surface roughening or other surface features to facilitate frictional engagement therebetween to prevent relative displacement between the replacement bodies 12 and 14 when the collar 16 is used to secure the second replacement body 14 in position relative to the first replacement body 12.

In the illustrated embodiment, the collet 18 includes a conically-shaped end portion 24 that defines an outer surface 25 which tapers outwardly away from a main segment 26 of the first replacement body 12 at an oblique angle relative to the longitudinal axis L. A plurality of kerf cuts or slits 28 extend inwardly from the end of the conical portion 24 and generally along the longitudinal axis L. The slits 28 form a plurality of individual fingers 30 that are capable of being elastically deformed or collapsed about the projecting portion 22 of the second replacement body 14. In the illustrated embodiment, the slits 28 extend along the axial length of the conical portion 24. However, in other embodiments, the slits 28 may extend beyond the conical portion 24 and along at least a portion of the main segment 26. The length of the slits 28 may be partially dependent on the material and wall thickness of the collet 18. In general, the slits 28 should have a length sufficient to allow for inward deformation of the fingers 30 and clamping or compression of the fingers 30 about the projecting portion 22.

As should be appreciated, the collet 18 forms a collar extending about the projecting portion 22 of the second replacement body 14. As set forth below, the collet 18 is capable of exerting a clamping force onto the projecting portion 22 of the second replacement body 14 when the collar 16 is axially displaced along the conical portion 24 of the collet 18. The first replacement body 12 also includes a threaded section 32 located adjacent the conical portion 24 of the collet 18. The threaded section 32 extends circumferentially about the outer surface of a portion of the main segment 26. As set forth in greater detail below, the threaded section 32 is used to threadably engage the collar 16 to the first replacement body 12.

In the illustrated embodiment, the main segment 26 of the first replacement body 12 includes a plurality of vascularization or bone growth apertures or windows 34 in communication with the hollow interior or axial passage 20. The apertures 34 are positioned about the periphery of the main segment 26 and may be provided in many configurations having various shapes and sizes. In one embodiment, the apertures 34 have a generally rectangular configuration. However, other configurations are also contemplated, including circular, elliptical or oval configuration, or any other configuration that would occur to one of skill in the art. The apertures 34 may be used to load bone growth promoting material such as bone graft (not shown) into the interior region or axial passage 20 of the first replacement body 12 and/or to facilitate vascularization or bone in-growth into the first replacement body 12 or other biological activity.

In the illustrated embodiment, the first replacement body 12 includes an end cap 36 positioned at the end of the collet 18 for engagement with the adjacent vertebral body or bone. As should be appreciated, the end cap 36 may be used to distribute loading over a greater surface area, thereby increasing stability of the device and inhibiting subsidence into the adjacent vertebral body or bone. However, it should be understood that other embodiments are also contemplated which do not include the end cap 36. In one embodiment, the end cap 36 is formed integral with the collet 18. However, in other embodiments, the end cap 36 may be formed separately from the collet 18 and attached thereto via any attachment technique know to those of skill in the art.

In the illustrated embodiment, the end cap 36 has a substantially smooth bone engagement surface. However, in other embodiments, the bone engagement surface of the end cap 36 may be provided with anchor elements, such as teeth, spikes, fins, peaks or other surface projections or surface roughening to facilitate gripping of the adjacent vertebral body or bone to prevent movement of the replacement body 12. Additionally, in the illustrated embodiment, the end cap 36 has a solid configuration and does not define any openings or apertures extending therethrough. However, in other embodiments, the end cap 36 may be provided with one or more openings or apertures extending through the bone engagement surface and in communication with the hollow interior or axial passage 20 to facilitate bone in-growth from the adjacent vertebral bodies or bone.

As indicated above, the second replacement body 14 includes a projecting portion or stem 22 that is sized and configured to be telescopically receiving within the axial passage 20 in the first replacement body 12. In the illustrated embodiment of the invention, the second replacement body 14 is provided with a hollow interior region 23 and includes a plurality of vascularization or bone growth apertures or windows 38 positioned about the periphery of the projecting portion 22 in communication with the interior region 23. Similar to the apertures 34 illustrated and described above with regard to the first replacement body 12, the apertures 38 may likewise be provided in many configurations having various shapes and sizes. The apertures 38 may be used to load bone growth promoting material such as bone graft (not shown) into the interior region 23 of the second replacement body 14 and/or to facilitate vascularization or bone in-growth into the second replacement body 14. In the illustrated embodiment, the second replacement body 14 is also provided with an end cap 40 configured similar to the end cap 36 illustrated and described above with regard to the first replacement body 12, and may also take on any of the configurations described above with regard to the end cap 36. However, it should be understood that other embodiments are also contemplated which do not include the end cap 40.

As indicated above, an osteogenetic or bone growth promoting material (not shown) may be loaded into either or both of the interior region 20 defined by the first replacement body and/or the interior region 23 defined by the second replacement body 14. Loading of the bone growth promoting material may occur prior to or subsequent to insertion of the expandable vertebral replacement device 10 into the intervertebral space. Additionally, the interior regions 20 and 23 may be filled with bone cement, therapeutic substances or other materials or substances that would occur to one of skill in the art.

Osteogenic or bone growth promoting materials which may be loaded into the interior regions 20 and 23 of the replacement bodies include, but are not limited to, bone morphogenic protein (BMP), bone graft material, bone chips or bone marrow, synthetic or natural autograft, allograft, xenograft, synthetic and natural bone graft substitutes such as bioceramics and polymers, osteoinductive factors, a demineralized bone matrix (DBM), mesenchymal stem cells, a LIM mineralization protein (LMP), or any other suitable bone growth promoting material or substance that would occur to one of skill in the art. Additionally, it should be understood that the bone growth promoting material may be used with or without a suitable carrier to aid in maintaining the material within the device. These carriers can include collagen-based carriers, bioceramic materials, such as BIOGLASS®, hydroxyapatite and calcium phosphate compositions. The carrier material may be provided in the form of a sponge, a block, folded sheet, putty, paste, graft material or other suitable forms. The bone growth promoting material may be provided in a composition which includes an effective amount of a bone morphogenetic protein (BMP), transforming growth factor β1, insulin-like growth factor 1, platelet-derived growth factor, fibroblast growth factor, LIM mineralization protein (LMP), and combinations thereof or other therapeutic or infection resistant agents. Additionally, the bone growth promoting material may be resorbable or non-resorbable. Examples of resorbable materials that may be used include, but are not limited to, polylactide, polyglycolide, tyrosine-derived polycarbonate, polyanhydride, polyorthoester, polyphosphazene, calcium phosphate, hydroxyapatite, bioactive glass, and various combinations thereof.

As set forth above, the projecting portion 22 of the second replacement body 14 is sized slightly smaller than the axial passage 20 defined by the first replacement body 12 to allow the projecting portion 22 to be telescopically received within the axial passage 20, thereby allowing the overall height h or axial length of the expandable vertebral replacement device 10 to be adjusted to a select height or axial length. The collar 16 is utilized to tighten or clamp the second replacement body 14 in a fixed or locked state relative to the first replacement body 12.

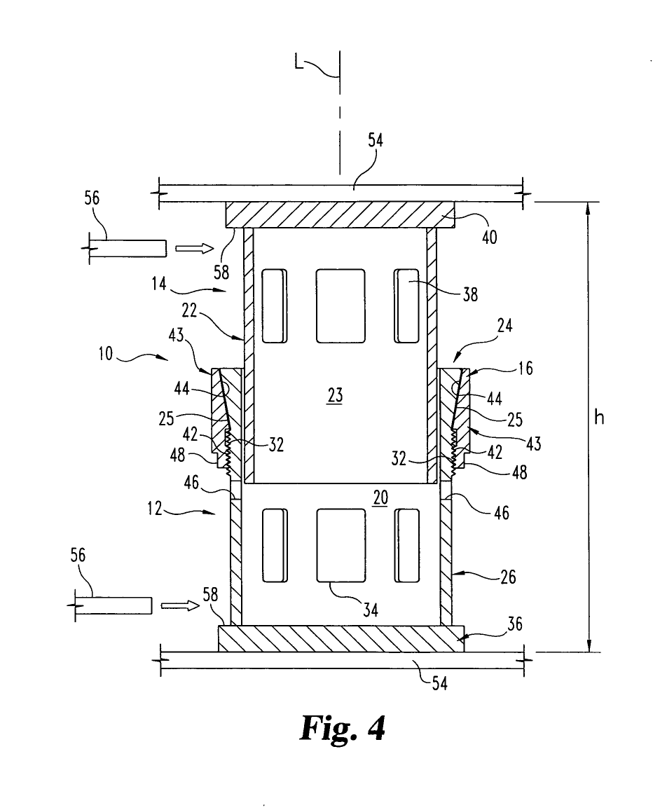

Referring to FIG. 4, shown therein is a cross-sectional view of the expandable vertebral replacement device 10. Once the projecting portion 22 of the second replacement body 14 is positioned within the axial passage 20 at the proper depth, the collar 16 is used to clamp the second replacement body 14 in position relative to the first replacement body 12. In the illustrated embodiment, the collar 16 includes an internally threaded section 42 that is configured to threadably engage the threaded section 32 extending about the main segment 26 of the first replacement body 12. The collar 16 also includes a tapering section 43 defining an inner surface 44 that tapers outwardly away from the threaded section 42 at an oblique angle relative to the longitudinal axis L. The tapering inner surface 44 of the collar 16 preferably corresponds to the tapering outer surface 25 defined by the conical portion 24 of the first replacement body 12.

As should be appreciated, the collar 16 is axially displaced generally along the longitudinal axis L by rotating the collar 16 so as to thread the collar 16 along the threaded segment 32 of the first replacement body 12. Axial displacement of the collar 16 in turn slidably engages the inner tapered surface 44 defined by the collar 16 against the outer tapered surface 25 defined by the conical portion 24 of the replacement body 12. Sliding displacement of the inner tapered surface 44 along the outer tapered surface 25 in turn applies pressure to the fingers 30 of the collet 18, thereby inwardly deforming or collapsing the fingers 30 toward the longitudinal axis L and clamping/compressing the fingers 30 about the projecting portion 22 of the second replacement body 14 to thereby secure the second replacement body 14 in a select axial position relative to the first replacement body 12.

Referring collectively to FIGS. 1 and 4, in the illustrated embodiment, the first replacement body 12 includes one or more key apertures 46 positioned about the periphery of the main segment 26 adjacent the threaded segment 32. Additionally, the collar 16 is provided with a bevel gear track 48 extending about at least a portion of the collar 16. The threaded segment 32 of the first replacement body 12 and the threaded section 42 of the collar 16 engage one another in a known manner. A bevel gear key 50 is provided which generally includes a distal end portion 52, a bevel gear 54 and a handle 56 to facilitate rotation of the gear key 50. The distal end portion 52 is sized for insertion into one of the key apertures 46 in the first replacement body 12, with the bevel gear 52 engaged with the bevel gear track 48 defined by the collar 16.

As should be appreciated, rotation of the bevel gear key 50 within the key aperture 46 correspondingly rotates the collar 16 about the longitudinal axis L, which in turn threadably engages the inner threads 42 defined by the collar 16 along the outer threads 32 defined by the first replacement body 12. Threading engagement between the inner thread 42 and the outer threads 32 results in axial displacement of the collar 16 generally along the longitudinal axis L, which in turn slidably engages the inner tapered surface 44 against the outer tapered surface 25, thereby inwardly deforming and clamping/compressing the fingers 30 about the projecting portion 22 of the second replacement body 14 to secure the second replacement body 14 in the select axial position relative to the first replacement body 12. As should be appreciated, rotating the bevel gear key 50 in the opposite direction would correspondingly axially displace the collar 16 in an opposite direction, thereby causing the collet 18 to release the second replacement body 14.

As should also be appreciated, as the collar 16 is axially displaced along the longitudinal axis L along the conical portion 24 of the first replacement body 12, the collar 16 will be axially displaced away from the key apertures 46 in the first replacement body 12 and the gear key 50. In order to maintain engagement between the bevel gear 52 and the bevel gear track 48 extending about the collar 16, as the collar 16 is displaced away from the gear key 50, the distal end portion 52 of the gear key 50 will be further inserted into the key aperture 46 in the first replacement body 12. Since the bevel gear 52 is tapered, engagement between the bevel gear 52 and the bevel gear track 48 will be maintained during axial displacement of the collar 16. Additionally, the components of the expandable vertebral replacement device 10 are preferably configured such that the collar 16 need only be rotated a relatively small amount to operably clamp the collet 18 of the first replacement body 12 about the projecting portion 22 of the second replacement body 14. For example, the inner diameter of the collet 18 and the conical portion 24 are preferably sized in relatively close tolerance with the outer diameter of the projecting portion 22, and the abutting tapered surfaces 25 and 44 defined by the collar 16 and the conical portion 24 are provided with a relatively large taper angle measured relative to the longitudinal axis L.

As illustrated in FIG. 4, once the expandable vertebral replacement device 10 is positioned within the intervertbral space between two respective vertebrae or bone structures 54, the arms 56 of a surgical distractor may be used to spread the first and second replacement bodies 12, 14 of the expandable vertebral replacement device 10 apart to a select overall height h. Various types and configurations of surgical distractors are contemplated for use in association with the present invention. In the illustrated embodiment, the arms 56 of the surgical distractor abuttingly engage the axially facing surfaces 58 defined by the end caps 36 and 40. However, it should be understood that the distractor may engaged other portions of the replacement bodies 12 and 14. Once the expandable vertebral replacement device 10 is adjusted to the select height h, the bevel gear key 50 is utilized to collapse the fingers 30 of the first replacement body 12 about the projecting portion 22 of the second replacement body 14 in the manner described above to secure the second replacement body 14 in position relative to the first replacement body 12 and to lock the expandable vertebral replacement device 10 at the select height h. If adjustments to the height h of the device 10 are necessary, the bevel gear key 50 may be used to loosen the expandable vertebral replacement device 10 in the manner described above.

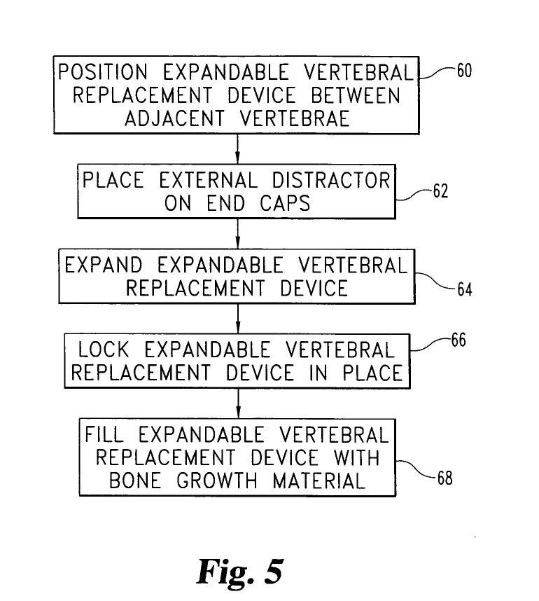

Referring collectively to FIGS. 4 and 5, the present invention also discloses a method of separating two adjacent vertebrae or bone structures 54. At step 60, an expandable vertebral replacement device 10, as disclosed herein, is positioned between first and second vertebrae 54 in a retracted or collapsed state. At step 62, the arms of a surgical distractor 56 are engaged with each of the replacement bodies 12 and 14, and more specifically to the axially facing surfaces 58 defined by the respective end caps 36, 40. At step 64, the expandable vertebral replacement device 10 is expanded to a select height between the first and second vertebrae 54 using the surgical distractor 56. Once expanded to the proper height, at step 66, a collar 16 of the expandable vertebral replacement device 10 is rotated to lock the expandable vertebral replacement device 10 at the select height between the first and second vertebrae 54.

While the present device has been described with respect to insertion of the vertebral replacement device within an intervertebral space between two vertebrae following removal of at least a portion of an intervening vertebra and the adjacent discs, it is contemplated that the device may be sized to span multiple vertebral levels to replace multiple vertebral bodies. Additionally, the vertebral replacement device may find application in other orthopedic areas outside of the spine.

Additionally, the size and shape of the vertebral replacement device may be selected to substantially match the implantation site. For example, while the present embodiment has been illustrated as having a substantially cylindrical configuration, it is also contemplated that in some applications, it may be desirable to provide the vertebral replacement device with a substantially D-shaped configuration such that the anterior portion of the device has an exterior convexly curved surface corresponding to the anterior of the vertebral body, while the posterior portion of the device is substantially flat or concave to allow the device to be positioned closer to the spinal canal without protruding into the spinal canal. In other embodiments, the outer profile of the vertebral replacement device may be provided with a rectangular or square configuration, an oval or elliptical configuration, a polygonal configuration, or any other configuration that would occur to one of skill in the art. Additionally, the shape of the vertebral replacement device may be configured to correspond to the shape of the adjacent vertebra or bone structure.

Embodiments of the vertebral replacement device, in whole or in part, may be formed from various types of biocompatible materials. Examples of biocompatible materials include, but are not limited to, non-reinforced polymers, carbon-reinforced polymer composites, PEEK and PEEK composites, shape-memory alloys, titanium, titanium alloys, cobalt chrome alloys, stainless steel, ceramics and combinations thereof. Additionally, radiographic markers may be located on the vertebral replacement device to provide the ability to monitor and determine radiographically or fluoroscopically the location of the device within the intervertebral space and relative to the remaining vertebral bodies. In some embodiments, radiographic markers may be used to show the location of the collar relative to the upper and lower replacement bodies, or to show the location of the upper and lower replacement bodies relative to one another.

In some embodiments, the vertebral replacement device or individual components of the device may be constructed of solid sections of bone or other tissues. In other embodiments, the vertebral replacement device may be constructed of planks of bone that are assembled into a final configuration. In some embodiments, the device may be constructed of planks of bone that are assembled along horizontal or vertical planes through one or more longitudinal axes of the device.

The surgical site may be accessed via any surgical approach that will allow adequate visualization and/or manipulation of the bone structures. For example, surgical approaches include, but are not limited to, any one or combination of anterior, antero-lateral, posterior, postero-lateral, transforaminal, and/or far lateral approaches. Additionally, insertion of the vertebral replacement device can occur through a single pathway or through multiple pathways, or through multiple pathways to multiple levels of the spinal column. Minimally invasive techniques using various types of instruments and devices are also contemplated.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. An expandable vertebral replacement device, comprising:
a first replacement body having a collet;
a second replacement body including a projecting portion telescopically received within said collet; and
a collar positioned about said first replacement body for engaging said collet to said second replacement body to thereby secure said second replacement body in a fixed position relative to said first replacement body.

2. The expandable vertebral replacement device of claim 1, wherein said collar includes a beveled gear track extending about at least a portion of said collar.

3. The expandable vertebral replacement device of claim 2, wherein said first replacement body includes at least one key aperture operable to rotatably receive an end portion of a bevel gear key, said bevel gear key including a bevel gear operably engaged with said bevel gear track on said collar.

4. The expandable vertebral replacement device of claim 3, wherein rotation of said bevel gear key within said key aperture results in rotation of said collar about said first replacement body, said rotation of said collar resulting in compression of said collet about said projecting portion of said second replacement body.

5. The expandable vertebral replacement device of claim 1, wherein said collar is threadably engaged with said first replacement body such that rotation of said collar causes said collet to compress down on said projecting portion of said second replacement body to fixedly engage said first replacement body in position relative to said second replacement body.

6. The expandable vertebral replacement device of claim 1, wherein said collar includes an inner tapered surface configured to engage an outer tapered surface of said collet; and
wherein axial displacement of said collar relative to said collet slidably engages said inner tapered surface along said outer tapered surface to compress said collet about said projecting portion of said second replacement body.

7. The expandable vertebral replacement device of claim 1, wherein said first and second replacement bodies each include a hollow interior region and at least one aperture communicating with said hollow interior region.

8. The expandable vertebral replacement device of claim 1, wherein each of said first and second replacement bodies include an end cap configured to engage adjacent bone.

9. The expandable vertebral replacement device of claim 1, wherein said collet includes a plurality of slits extending from an end thereof to facilitate inward deformation of said collet to compress said collet about said projecting portion of said second replacement body.

10. An expandable vertebral replacement device, comprising:
a first cylindrical replacement body having an upper portion and a lower portion defining at least a partially hollow interior area, wherein said upper portion includes a collet;
a second cylindrical replacement body including a projecting portion positioned within said collet of said first cylindrical replacement body; and
a collar positioned about said upper portion of said first cylindrical replacement body for fixedly engaging said collet to said projecting portion of said second cylindrical replacement body to maintain said second cylindrical replacement body in a fixed position relative to said first cylindrical replacement body.

11. The expandable vertebral replacement device of claim 10, wherein said upper portion of said first cylindrical replacement body includes a threaded section.

12. The expandable vertebral replacement device of claim 11, wherein said collar includes a threaded section and a tapered section.

13. The expandable vertebral replacement device of claim 12, wherein said threaded section of said collar engages said threaded section of said first cylindrical replacement body.

14. The expandable vertebral replacement device of claim 13, wherein as said collar is tightened on said first cylindrical replacement body, said tapered section forces said collet to forcibly clamp down on said projecting portion of said second cylindrical replacement body.

15. The expandable vertebral replacement device of claim 10, wherein said collar includes an internal threaded section for engaging an external threaded section located on said upper portion of said first cylindrical replacement body.

16. The expandable vertebral replacement device of claim 10, wherein a lower surface of said collar includes a bevel gear track extending about at least a portion of said collar.

17. The expandable vertebral replacement device of claim 16, wherein said upper portion of said first cylindrical replacement body includes at least one key aperture for rotatably receiving an end portion of a bevel gear key, said bevel gear key including a bevel gear operably engaged with said bevel gear track on said collar.

18. The expandable vertebral replacement device of claim 17, wherein rotation of said bevel gear key within said key aperture results in rotation of said collar about said first cylindrical replacement body, said rotation of said collar resulting in compression of said collet about said projecting portion of said second cylindrical replacement body.

19. The expandable vertebral replacement device of claim 10, wherein said first and second cylindrical replacement bodies each include an end cap configured to engage adjacent bone.

20. The expandable vertebral replacement device of claim 10, wherein each of said first and second cylindrical replacement bodies includes a hollow interior region and at least one aperture communicating with said hollow interior region.

21. The expandable vertebral replacement device of claim 10, wherein said collet includes a conical shaped outer surface.

22. The expandable vertebral replacement device of claim 21, wherein said collar includes an inner tapered surface positioned in contact said conical shaped outer surface of said collet, wherein axial displacement of said collar causes said inner tapered surface to slidably engage said conical shaped outer surface to compress said collet about said projecting portion of said second cylindrical replacement body.

23. A method of supporting two adjacent vertebrae, comprising the steps of:
placing an expandable vertebral replacement device between the adjacent vertebrae;
positioning an external distractor into engagement with opposite end portions of the expandable vertebral replacement device;
expanding the expandable vertebral replacement device along a longitudinal axis to a select height between the first and second vertebrae with the external distractor; and
rotating a collar portion of the expandable vertebral replacement device about the longitudinal axis to lock the expandable vertebral replacement device at the select height.

24. The method of claim 23, wherein the expandable vertebral replacement device comprises a first replacement body and a second replacement body, wherein a projecting portion of the second replacement body is telescopically received within an axial passage defined by the first replacement body.

25. The method of claim 24, wherein the first replacement body includes a collapsible collet defining the axial passage, and wherein the projecting portion of the second replacement body is telescopically positioned within the axial passage.

26. The method of claim 25, wherein the collar is threadably engaged with an upper portion of the first replacement body to axially displace the collar along the longitudinal axis to collapse the collet about the projecting portion of the second replacement body.

27. The method of claim 26, wherein the collar includes a tapered section that forces the collet against the projecting portion of the second replacement body during axial displacement of the collar along the longitudinal axis to thereby locking the expandable vertebral replacement device at the select height.

28. The method of claim 23, further comprising the step of rotatably engaging a bevel gear key within a key aperture defined by the expandable vertebral replacement device such that a gear portion of the bevel gear key is operably engaged with a bevel gear track extending about the collar, and wherein rotation of the bevel gear key results in corresponding rotation of the collar about the longitudinal axis and axial displacement of the collar along the longitudinal axis.

29. The method of claim 23, wherein the expandable vertebral replacement device includes a hollow interior region and at least one aperture extending through the expandable vertebral replacement device and in communication with the hollow interior region.

30. The method of claim 29, further comprising inserting a bone growth promoting material into the hollow interior region of the expandable vertebral replacement device.
